SEQUENCE LISTING (6 sequences)

SEQ ID NO: 1 - DNA, 804 bp, Homo sapiens
atgaaagctgcggtgctgaccttggccgtgctcttcctgacggggagccaggctcggcatttctggcagcaagatgaacccccccagagcccctgggatcgagtgaaggacctggccactgtgtacgtggatgtgctcaaagacagcggcagagactatgtgtcccagtttgaaggctccgccttgggaaaacagctaaacctaaagctccttgacaactgggacagcgtgacctccaccttcagcaagctgcgcgaacagctcggccctgtgacccaggagttctgggataacctggaaaaggagacagagggcctgaggcaggagatgagcaaggatctggaggaggtgaaggccaaggtgcagccctacctggacgacttccagaagaagtggcaggaggagatggagctctaccgccagaaggtggagccgctgcgcgcagagctccaagagggcgcgcgccagaagctgcacgagctgcaagagaagctgagcccactgggcgaggagatgcgcgaccgcgcgcgcgcccatgtggacgcgctgcgcacgcatctggccccctacagcgacgagctgcgccagcgcttggccgcgcgccttgaggctctcaaggagaacggcggcgccagactggccgagtaccacgccaaggccaccgagcatctgagcacgctcagcgagaaggccaagcccgcgctcgaggacctccgccaaggcctgctgcccgtgctggagagcttcaaggtcagcttcctgagcgctctcgaggagtacactaagaagctcaacacccagtga

SEQ ID NO: 2 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 3 - DNA, 20 bp, Homo sapiens
tggatgtgctcaaagacagc

SEQ ID NO: 4 - DNA, 20 bp, Homo sapiens
acctcctccagatccttgct

SEQ ID NO: 5 - DNA, 20 bp, Murine
tggatgcggtcaaagacagc

SEQ ID NO: 6 - DNA, 20 bp, Murine
acttcctctaggtccttgtt

(19) United States
(12) Patent Application Publication
(10) Pub. No.: US 2013/0317093 A1
(43) Pub. Date: Nov. 28, 2013

(54) METHODS OF TREATING ALZHEIMER'S DISEASE WITH APO A-1 MILANO

(71) Applicant: Prediman K. Shah, Los Angeles, CA (US)

(72) Inventor: Prediman K. Shah, Los Angeles, CA (US)

(73) Assignee: CEDARS-SINAI MEDICAL CENTER, Los Angeles, CA (US)

(21) Appl. No.: 13/983,956

(22) PCT Filed: Feb. 9, 2012
(86) PCT No.: PCT/US12/24525

§ 371 Date: Aug. 6, 2013

Related U.S. Application Data
(60) Provisional application No. 61/441,601, filed on Feb. 10, 2011.

(51) Int. Cl. A61K 31/7088

(52) U.S. Cl. CPC A61K 31/7088
USPC 514/44 R

(57) ABSTRACT

This invention relates to the treatment of Alzheimer's disease, the reduction of the progression of Alzheimer's disease, and the alleviation of symptoms of Alzheimer's disease by administering ApoA-1 Milano based therapies to provide anti-inflammatory, antioxidant, and lipid depleting effects to brain tissue. In particular embodiments, the method comprises administering a composition comprising an rAAV vector encoding ApoA-1 Milano or an active fragment thereof, to a mammal having Alzheimer's disease or a symptom of Alzheimer's disease.

### FIELD OF INVENTION

This invention relates to the treatment of Alzheimer's disease and providing anti-inflammatory, antioxidant, and lipid depleting effects to brain tissue.

### BACKGROUND

All publications herein are incorporated by reference to the same extent as if each individual publication or patent application was specifically and individually indicated to be incorporated by reference. The following description includes information that may be useful in understanding the present invention, it is not an admission that any of the information provided herein is prior art or relevant to the presently claimed invention, or that any publication specifically or implicitly referenced is prior art Alzheimer's disease has been an irreversible, progressive brain disease that slowly destroys memory and thinking skills, and eventually even the ability to carry out the simplest tasks. Two abnormal structures called plaques and tangles are prime suspects in damaging and killing nerve cells. Plaques are deposits of a protein fragment called beta-amyloid that build up in the spaces between nerve cells. Tangles are twisted fibers of another protein called tau that builds up inside cells. Though most people develop some plaques and tangles as they age, those with Alzheimer's tend to develop far more. They also tend to develop them in a predictable pattern, beginning in areas important for memory before spreading to other regions.

Research on how the brain changers normally with age is shedding light as scientists are learning how age-related changes in the brain may harm neurons and contribute to Alzheimer's damage. These age-related changes include atrophy of certain parts of the brain, inflammation, and the production of unstable molecules called free radicals.

Research into the pathology of Alzheimer's disease has uncovered that plaque is found at significantly greater levels in the brain and its surrounding tissue in individuals with the disease relative to the healthy population. In fact, individuals with Alzheimer's disease produce certain amyloidogenic peptides in the brain, resulting in the formation of amyloid plaques. These plaques are believed to be the fundamental problem in Alzheimer's disease; they interfere with brain function, causing the memory loss and deterioration of other cognitive abilities typically associated with Alzheimer's disease.

Among other things, cholesterol is related to the formation of plaque in the vasculature and in other physiological regions of the body. In fact, it is believed that brain cell cholesterol content can regulate the production of the aforementioned amyloid plaques that are deposited in the brains of those with Alzheimer's disease at an abnormally high quantity. Cholesterol levels can be modulated by high density lipoprotein (“HDL”)-based therapies.

Apolipoprotein A-1 (“ApoA-1”) ApoA-1, a major component of high density lipoprotein (“HDL”), has been shown to have anti-atherogenic properties. An Arg173 to Cys point mutation known as ApoA-1 Milano has demonstrated efficacy in both the prevention and treatment of atherosclerotic lesions in murine and rabbit animal models, with potency greater than that of wild type ApoA-I. A human trial testing recombinant ApoA-1 Milano infusions has similarly shown significant and rapid reduction in coronary atheroma volumes. It has thus been shown that therapies incorporating ApoA-1 Milano for modulating the activity of these genes or the levels of their products may be effective in reducing atherosclerotic plaque development.

Despite the progress of research, there remains a need in the art for treatments for Alzheimer's disease, including providing anti-inflammatory, antioxidant, and lipid depleting effects to brain tissue to treat or slow down the progression of the disease.

### SUMMARY OF THE INVENTION

The following embodiments and aspects thereof are described and illustrated in conjunction with compositions and methods which are meant to be exemplary and illustrative, not limiting in scope.

Various embodiments of the present invention provide for a method, comprising providing a composition comprising an rAAV vector encoding ApoA-1 Milano or an active fragment thereof; and administering the composition to a mammal in need of treatment for Alzheimer's disease, in need of slowing the progression of Alzheimer's disease, or in need of alleviating a symptom of Alzheimer's disease, to deliver the ApoA-1 Milano or an active fragment thereof to brain tissue, to treat the Alzheimer's disease, to slow the progression of Alzheimer's disease, or to alleviate the symptom of Alzheimer's disease.

In various embodiments, the mammal can be in need of treatment for Alzheimer's disease and the method can treat Alzheimer's disease. In certain embodiments, the mammal can be in need of slowing the progression of Alzheimer's disease and the method can slow the progression of Alzheimer's disease. In certain embodiments, the mammal can be in need of alleviating the symptoms of Alzheimer's disease and the method can alleviate a symptom of Alzheimer's disease. In certain embodiments, administering the composition can result in the delivery of the ApoA-1 Milano gene or a gene encoding an active fragment of ApoA-1 Milano to brain tissue. In certain embodiments, administering the composition can result in the secretion of ApoA-1 Milano or an active fragment thereof directly into the circulation of the mammal.

In various embodiments, the rAAV vector can be an rAAV8 vector encoding ApoA-1 Milano or an active fragment thereof. In certain embodiments, the rAAV vector can be an rAAV2 vector encoding Apo A-1 Milano or an active fragment thereof. In certain embodiments, the rAAV vector can be an rAAV9 vector encoding ApoA-1 Milano or an active fragment thereof.

In various embodiments, administering the composition can be by intravenous injection. In certain embodiments, administering the composition can be by intramuscular injection.

In various embodiments, the composition can comprise an rAAV8 vector encoding ApoA-1 Milano or an active fragment thereof, and administering the composition to the mammal can be by intravenous injection. In certain embodiments, the composition can comprise an rAAV9 vector encoding ApoA-1 Milano or an active fragment thereof, and administering the composition to the mammal can be by intravenous injection.

Various embodiments of the present invention provide for a method of delivering ApoA1-M to brain tissue of a mammal having Alzheimer's disease or a symptom of Alzheimer's disease, comprising providing a composition comprising an rAAV vector encoding ApoA-1 Milano or an active fragment thereof; and administering the composition to a mammal having Alzheimer's disease or a symptom of Alzheimer's disease to deliver the ApoA-1 Milano or an active fragment thereof to the brain tissue of the mammal.

In various embodiments, the rAAV vector can be an rAAV8 vector encoding ApoA-1 Milano or an active fragment thereof. In certain embodiments, the rAAV vector can be an rAAV2 vector encoding Apo A-1 Milano or an active fragment thereof. In certain embodiments, the rAAV vector can be an rAAV9 vector encoding ApoA-1 Milano or an active fragment thereof.

In various embodiments, administering the composition is by intravenous injection. In certain embodiments, administering the composition is by intramuscular injection.

Other features and advantages of the invention will become apparent from the following detailed description, taken in conjunction with the accompanying drawings, which illustrate, by way of example, various features of embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 depicts real-time PCT Quantitative analysis of APO A-1 Milano mRNA expression in mice tissues. Values were normalized against GAPDH mRNA. Data showed a significantly higher level of APO A-1 Milano. rAAV8 mediated transgene expression compared to rAAV2 in the brain (11.85±2.4 vs. 0.95±0, p<0.05), heart 102.3±24.20 vs. 0.9±0.5, p<0.001), liver (32.14±14.56 vs. 1.37±0.22, p=0.05), lung (16.49±10.75 vs. 1.86±1.8, p=0.25), spleen (5.41±1.59 vs. 3.39±1.69, p=0.22) and kidney (1.96±0.8 vs. 0.81±0.018, p=0.119).

FIG. 2 depicts qPCR for ApoA1M expression in the brain of rAAV injected B6cg mice in accordance with various embodiment of the present invention.

FIG. 3 depicts ApoA1-M expression for various rAAV vectors in the brain by q-PCR in accordance with various embodiments of the present invention.

FIG. 4 depicts human ApoA1-M (A1M) plasma levels for various rAAV vectors in B6cg mice in accordance with various embodiments of the present invention.

FIG. 5 depicts human ApoA1-M (A1M) plasma levels for various rAAV vectors in B6cg mice in accordance with various embodiments of the present invention.

FIG. 6 depicts human ApoA1-M (A1M) plasma in female versus male (ng/ml) in accordance with various embodiments of the present invention.

FIG. 7 depicts a gene construct used in various embodiments of the present invention. The Apo AI-M (AIM) gene includes the secretion signal.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., J. Wiley & Sons (New York, N.Y. 2001); March, Advanced Organic Chemistry Reactions, Mechanisms and Structure 5^{th} ed., J. Wiley & Sons (New York, N.Y. 2001); and Sambrook and Russel, Molecular Cloning: A Laboratory Manual 3rd ed., Cold Spring Harbor Laboratory Press (Cold Spring Harbor, N.Y. 2001), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

“Beneficial results” may include, but are in no way limited to, lessening or alleviating the severity of the disease condition, preventing the disease condition from worsening, reducing the likelihood of the disease condition worsening, curing the disease condition and prolonging a patient's life or life expectancy.

“Gene transfer” or “gene delivery” refers to methods or systems for reliably inserting foreign DNA into host cells. Such methods can result in transient expression of non-integrated transferred DNA, extrachromosomal replication and expression of transferred replicons (e.g., episomes), or integration of transferred genetic material into the genomic DNA of host cells. Gene transfer provides a unique approach for the treatment of acquired and inherited diseases. A number of systems have been developed for gene transfer into mammalian cells. See, e.g., U.S. Pat. No. 5,399,346.

“Vector” refers to any genetic element, such as a plasmid, phage, transposon, cosmid, chromosome, virus, virion, etc., which is capable of replication when associated with the proper control elements and which can transfer gene sequences between cells. Thus, the term includes cloning and expression vehicles, as well as viral vectors.

“AAV vector” refers to any vector derived from any adeno-associated virus serotype, including, without limitation, AAV-1, AAV-2, AAV-3, AAV-4, AAV-5, AAV-7, AAV-8, AAV-9, and AAV-10 and the like. AAV vectors can have one or more of the AAV wild-type genes deleted in whole or in part, preferably the Rep and/or Cap genes, but retain functional flanking ITR sequences. Functional ITR sequences are generally necessary for the rescue, replication, packaging and potential chromosomal integration of the AAV genome. Thus, an AAV vector is defined herein to include at least those sequences required in cis for replication and packaging (e.g., functional ITRs) of the virus. The ITRs need not be the wild-type nucleotide sequences, and may be altered (e.g., by the insertion, deletion or substitution of nucleotides) so long as the sequences provide for functional rescue, replication and packaging.

“Recombinant virus” refers to a virus that has been genetically altered (e.g., by the addition or insertion of a heterologous nucleic acid construct into the particle).

“AAV virion” refers to a complete virus particle, such as a wild-type (“wt”) AAV virus particle (i.e., including a linear, single-stranded AAV nucleic acid genome associated with an AAV capsid protein coat). In this regard, single-stranded AAV nucleic acid molecules of either complementary sense (i.e., “sense” or “antisense” strands) can be packaged into any one AAV virion; both strands are equally infectious. In addition, the AAV capsid protein coat can be from any of the various AAV serotypes depending on the target of the AAV virion.

A “recombinant AAV virion” or “rAAV virion” is defined herein as an infectious, replication-defective virus composed of an AAV protein shell, encapsidating a heterologous DNA molecule of interest (e.g., genes encoding ApoA-1 Milano) which is flanked on both sides by AAV ITRs. A rAAV virion may be produced in a suitable host cell which has had an AAV vector. AAV Rep and Cap functions and helper virus functions introduced therein. In this manner, the host cell is rendered capable of producing AAV replication and capsid proteins that are required for replicating and packaging the AAV vector (i.e., containing a recombinant nucleotide sequence of interest) into recombinant virion particles for subsequent gene delivery. The complete transgene may consist of a promoter, the coding sequences, usually a cDNA and a polyadenylation signal. A transgene may also include regulatory sequences and intron regions. Promoters that would regulate transgene expression may include constitutive, inducible and tissue-specific promoters.

The term “transfection” is used herein to refer to the uptake of foreign DNA by a cell. A cell has been “transfected” when exogenous DNA has been introduced inside the cell membrane. A number of transfection techniques are generally known in the art. See, e.g., Graham et al. (1973) Virology, 52:456, Sambrook et al. (1989) Molecular Cloning, a laboratory manual, Cold Spring Harbor Laboratories, New York, Davis et al. (1986) Basic Methods in Molecular Biology, Elsevier, and Chu et al. (1981) Gene 13:197. Such techniques can be used to introduce one or more exogenous DNA moieties, such as a plasmid vector and other nucleic acid molecules, into suitable host cells. The term refers to both stable and transient uptake of the genetic material.

The term “transduction” denotes the delivery of a DNA molecule to a recipient cell either in vivo or in vitro, via any method of gene delivery, including replication-defective viral vectors, such as via a rAAV.

The term “heterologous,” as it relates to nucleic acid sequences such as gene sequences and control sequences, denotes sequences that are not normally joined together and/or are not normally associated with a particular virus. Allelic variation or naturally occurring mutational events do not give rise to heterologous DNA, as used herein.

“DNA” is meant to refer to a polymeric form of deoxyribonucleotides (i.e., adenine, guanine, thymine and cytosine) in double-stranded or single-stranded form, either relaxed or supercoiled. This term refers only to the primary and secondary structure of the molecule, and does not limit it to any particular tertiary forms. Thus, this term includes single- and double-stranded DNA found, inter alia, in linear DNA molecules (e.g., restriction fragments), viruses, plasmids, and chromosomes. In discussing the structure of particular DNA molecules, sequences may be described herein according to the normal convention of giving only the sequence in the 5′ to 3′ direction along the non-transcribed strand of DNA (i.e., the strand having the sequence homologous to the mRNA). The term captures molecules that include the four bases adenine, guanine, thymine and cytosine, as well as molecules that include base analogues which are known in the art.

A “gene” or “coding sequence” or a sequence which “encodes” a particular protein is a nucleic acid molecule that is transcribed (in the case of DNA) and translated (in the case of mRNA) into a polypeptide in vitro or in vivo when placed under the control of appropriate regulatory sequences: although one of skill in the art will readily appreciate that various polynucleotides do not operate in this fashion (e.g., antisense RNA, siRNA, ribozymes, wherein the RNA transcript is the product). With respect to protein products (i.e., not RNA products), the boundaries of the coding sequence are determined by a start codon at the 5′ (i.e., amino) terminus and a translation stop codon at the 3′ (i.e., carboxy) terminus. A gene can include, but is not limited to, cDNA from prokaryotic or eukaryotic mRNA, genomic DNA sequences from prokaryotic or eukaryotic DNA, and even synthetic DNA sequences. A transcription termination sequence will usually be located 3′ to the gene sequence. Moreover, a “gene” (i) starts with a promoter region containing multiple regulatory elements, possibly including enhancers, for directing transcription of the coding region sequences; (ii) includes coding sequences, which start at the transcriptional start site that is located upstream of the translational start site and ends at the transcriptional stop site, which may be quite a bit downstream of the stop codon (a polyadenylation signal is usually associated with the transcriptional stop site and is located upstream of the transcriptional stop); and (iii) may contain introns and other regulatory sequences to modulate expression and improve stability of the RNA transcript.

The term “control elements” refers collectively to promoter regions, polyadenylation signals, transcription termination sequences, upstream regulatory domains, origins of replication, internal ribosome entry sites (“IRES”), enhancers, and the like, which collectively provide for the replication, transcription and translation of a coding sequence in a recipient cell. Not all of these control elements need always be present, so long as the selected coding sequence is capable of being replicated, transcribed and translated in an appropriate host cell.

The term “promoter region” is used herein in its ordinary sense to refer to a nucleotide region including a DNA regulatory sequence, wherein the regulatory sequence is derived from a gene which is capable of binding RNA polymerase and initiating transcription of a downstream (3′-direction) coding sequence.

“Operably linked” refers to an arrangement of elements wherein the components so described are configured so as to perform their usual function. Thus, control elements operably linked to a coding sequence are capable of effecting the expression of the coding sequence. The control elements need not be contiguous with the coding sequence, so long as they function to direct the expression thereof. Thus, for example, intervening untranslated yet transcribed sequences can be present between a promoter sequence and the coding sequence and the promoter sequence can still be considered “operably linked” to the coding sequence.

For the purpose of describing the relative position of nucleotide sequences in a particular nucleic acid molecule throughout the instant application, such as when a particular nucleotide sequence is described as being situated “upstream,” “downstream,” “5′,” or “3′” relative to another sequence, it is to be understood that it is the position of the sequences in the non-transcribed strand of a DNA molecule that is being referred to as is conventional in the art.

“Homology” and “homologous” as used herein refer to the percent of identity between two polynucleotide or two polypeptide moieties. The correspondence between the sequences from one moiety to another can be determined by techniques known in the art. For example, homology can be determined by a direct comparison of the sequence information between two polypeptide molecules by aligning the sequence information and using readily available computer programs. Alternatively, homology can be determined by hybridization of polynucleotides under conditions which form stable duplexes between homologous regions, followed by digestion with single-stranded-specific nuclease(s), and size determination of the digested fragments. Two DNA or two polypeptide sequences are “substantially homologous” to each other when at least about 80%, preferably at least about 90%, and most preferably at least about 95% of the nucleotides or amino acids, respectively, match over a defined length of the molecules, as determined using the methods above.

“Isolated” as used herein when referring to a nucleotide sequence, vector, etc., refers to the fact that the indicated molecule is present in the substantial absence of other biological macromolecules of the same type. Thus, an “isolated nucleic acid molecule which encodes a particular polypeptide” refers to a nucleic acid molecule that is substantially free of other nucleic acid molecules that do not encode the subject polypeptide. Likewise, an “isolated vector” refers to a vector that is substantially free of other vectors that differ from the subject vector. However, the subject molecule or vector may include some additional bases or moieties that do not deleteriously affect the basic characteristics of the composition.

“Purified” as used herein when referring to a vector, refers to a quantity of the indicated vector that is present in the substantial absence of other biological macromolecules. Thus, a “purified vector” refers to a composition that includes at least 80% subject vector, preferably at least 90%, 91%, 92%, 93%, 94%, 95%, 96%, 97%. 98% or 99% subject vector with respect to other components of the composition

“Mammal” as used herein refers to any member of the class Mammalia, including, without limitation, humans and nonhuman primates such as chimpanzees and other apes and monkey species; farm animals such as cattle, sheep, pigs, goats and horses, domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be included within the scope of this term.

The present invention includes compositions and methods for the treatment of Alzheimer's disease using ApoA-1 Milano based therapies. More specifically, the invention includes a method of treating Alzheimer's disease and reducing the progression of Alzheimer's disease by administering a therapeutically effective amount of an ApoA-1 Milano based therapy to a mammal. The invention also includes a method of delivering ApoA-1 Milano to brain tissue of a mammal having Alzheimer's disease. In various embodiments of the present invention, the mammal is a human. While not wishing to be bound by any particular theory, it is believed that ApoA-1 Milano based therapies, which are known to be effective in modulating atherosclerotic plaque development and persistence, can be effective in the treatment of Alzheimer's disease by lowering the levels of amyloid plaques in the brain and/or by preventing the additional build-up thereof, and/or through its anti-inflammatory effects including favorably modifying the phenotype of circulating and/or brain macrophages or macrophage-like cells from a pro-inflammatory to an anti-inflammatory or less inflammatory phenotype. One of ordinary skill in the art will readily appreciate that these effects are beneficial to the treatment of Alzheimer's disease. It is further believed that the anti-inflammatory, antioxidant, and lipid depleting effects of ApoA-1 Milano can be effective in the treatment of Alzheimer's disease and reduce the progression of Alzheimer's disease.

The ApoA-1 Milano based therapy may be administered by any appropriate technique, as will be readily appreciated by those of skill in the art. With respect to embodiments of the present invention that incorporate ApoA-1 Milano therapeutics, the therapy may be administered by a gene therapeutic approach. For instance, rAAV virions including heterologous DNA corresponding to an ApoA-1 Milano coding sequence may be generated by any conventional technique known in the art. By way of example, the recombinant AAV virions of the present invention, including the ApoA-1 Milano DNA of interest, can be produced by a standard methodology that generally involves the steps of: (1) introducing an AAV vector into a host cell; (2) introducing an AAV helper construct into the host cell, where the helper construct includes AAV coding regions capable of being expressed in the host cell to complement AAV helper functions missing from the AAV vector; (3) introducing one or more helper viruses and/or accessory function vectors into the host cell, wherein the helper virus and/or accessory function vectors provide accessory functions capable of supporting efficient rAAV virion production in the host cell; and (4) culturing the host cell to produce rAAV virions. The AAV vector, AAV helper construct and the helper virus or accessory function vector(s) can be introduced into the host cell either simultaneously or serially, using standard transfection techniques.

AAV vectors are constructed using known techniques to at least provide, as operatively linked components in the direction of transcription, (a) control elements including a transcriptional initiation region, (b) the ApoA-1 Milano DNA of interest and (c) a transcriptional termination region. Moreover, any coding sequence sufficiently homologous to the ApoA-1 Milano coding sequence so as to exhibit functional properties substantially similar to the ApoA-1 Milano coding sequence can be used in connection with alternate embodiments of the present invention. The control elements are selected to be functional in the targeted cell(s). The resulting construct, which contains the operatively linked components, may be bounded (5′ and 3′) with functional AAV ITR sequences. The nucleotide sequences of AAV ITR regions are known. See, e.g., Kotin, R. M. (1994) Human Gene Therapy 5:793-801; Berns, K. I. “Parvoviridae and their Replication” in Fundamental Virology. 2^{nd} Edition, (B. N. Fields and D. M. Knipe, eds.) for the AAV-2 sequence. AAV ITRs used in the vectors of the invention need not have a wild-type nucleotide sequence, and may be altered (e.g., by the insertion, deletion or substitution of nucleotides). Additionally, AAV ITRs may be derived from any of several AAV serotypes, including, without limitation, AAV-1, AAV-2, AAV-3, AAV-4, AAV-5, AAVX7, AAV-8, AAV-9, AAV-10 and the like. Furthermore, 5′ and 3′ ITRs that flank a selected nucleotide sequence in an AAV expression vector need not necessarily be identical or derived from the same AAV serotype or isolate, so long as they function as intended (i.e., to allow for excision and replication of the bounded ApoA-1 Milano nucleotide sequence of interest).

Therefore, in accordance with an embodiment of the invention, the rAAV virions including an ApoA-1 Milano coding sequence are delivered to a mammal in a sufficient quantity and by a sufficient delivery route so as to effect gene transfer. This may provide an effective treatment for Alzheimer's disease in the mammal.

Accordingly, various embodiments of the present invention provide for a method of delivering ApoA-1 Milano to brain tissue of a mammal having Alzheimer's disease or a symptom of Alzheimer's disease, comprising: providing a composition comprising an rAAV vector encoding ApoA-1 Milano or an active fragment thereof; and administering the composition to the mammal.

Various embodiments of the present invention provide for a method of slowing the progression of Alzheimer's disease in a mammal having Alzheimer's disease or a symptom of Alzheimer's disease, comprising: providing a composition comprising an rAAV vector encoding ApoA-1 Milano or an active fragment thereof: and administering the composition to the mammal.

Various embodiments of the present invention provide for a method of treating Alzheimer's disease in a mammal having Alzheimer's disease or a symptom of Alzheimer's disease, comprising: providing a composition comprising an rAAV vector encoding ApoA-1 Milano or an active fragment thereof; and administering the composition to the mammal.

In various embodiments, the rAAV vector encoding ApoA-1 Milano is rAAV8 vector encoding ApoA-1 Milano or an active fragment thereof. In various embodiments, the rAAV vector encoding ApoA-1 Milano is rAAV2 vector encoding Apo A-1 Milano or an active fragment thereof. In various embodiments, the rAAV vector encoding ApoA-1 Milano is rAAV9 vector encoding ApoA-1 Milano or an active fragment thereof.

In various embodiments, administering the composition comprises administering by intravenous injection. In various embodiments, administering the composition comprises administering by intramuscular injection. These methods result in the delivery of the ApoA-1 Milano gene or a gene encoding an active fragment of ApoA-1 Milano to brain tissue. These methods also result in secretion of ApoA-1 Milano or an active fragment thereof directly into the circulation.

In particular embodiments, the method of delivering ApoA-1 Milano to brain tissue of a mammal having Alzheimer's disease comprises providing a composition comprising an rAAV8 vector encoding ApoA-1 Milano or an active fragment thereof: and administering the composition to the mammal via intravenous injection.

In particular embodiments, the method of slowing the progression of Alzheimer's disease in a mammal having Alzheimer's disease comprises: providing a composition comprising an rAAV8 vector encoding Apo A-1 Milano or an active fragment thereof; and administering the composition to the mammal via intravenous injection.

In particular embodiments, the method of treating Alzheimer's disease in a mammal having Alzheimer's disease comprises: providing a composition comprising an rAAV8 vector encoding ApoA-1 Milano or an active fragment thereof; and administering the composition to the mammal via intravenous injection.

In particular embodiments, the method of delivering ApoA-1 Milano to brain tissue of a mammal having Alzheimer's disease comprises providing a composition comprising an rAAV9 vector encoding ApoA-1 Milano or an active fragment thereof; and administering the composition to the mammal via intravenous injection.

In particular embodiments, the method of slowing the progression of Alzheimer's disease in a mammal having Alzheimer's disease comprises: providing a composition comprising an rAAV9 vector encoding Apo A-1 Milano or an active fragment thereof, and administering the composition to the mammal via intravenous injection.

In particular embodiments, the method of treating Alzheimer's disease in a mammal having Alzheimer's disease comprises: providing a composition comprising an rAAV9 vector encoding ApoA-1 Milano or an active fragment thereof; and administering the composition to the mammal via intravenous injection.

An active fragment of ApoA-1 Milano gene refers to a nucleotide sequence that encodes a fragment of ApoA-1 Milano that retains the same or substantially the same biological activity of ApoA-1 Milano protein with respect to its anti-inflammatory, antioxidant and/or lipid depleting effects. An active fragment of ApoA-1 Milano protein refers to an amino acid sequence that retains the same or substantially the same biological activity of ApoA-1 Milano protein with respect to its anti-inflammatory, antioxidant and/or lipid depleting effects.

In various embodiments the rAAV vector is produced by the process of: (i) providing a first plasmid that comprises ApoA-1 Milano or a fragment thereof, (ii) providing a second plasmid that is complementary to the first plasmid and which comprises components for rescue and packaging, (iii) co-transfecting the first and second plasmids into a host cell, and (iv) generating a quantity of said rAAV vector from said co-transfected host cell, wherein the pair of said first and second plasmids is selected such that said rAAV vector is targeted for delivery to a specific tissue type.

In various embodiments, the second plasmid further comprises AAV rescue and packaging components derived from an AAV serotype selected from the group consisting of AAV1, AAV2, AAV5, AAV7, AAV8, AAV9, AAV10 and combinations thereof.

In various embodiments, the vectors of the present invention are based on the vector described in U.S. Pat. No. 5,474,935, with the transgene being ApoA-1 Milano. Preparation of rAAV vectors can be as described in Chatterjee, S. & K. K. Wong, Adeno-associated virus vectors for the delivery of ribozymes. In “Intracellular Ribozyme Applications: Principles and Protocols,” J J Rossi and L. Couture (Eds.), Horizon Scientific Press, pp. 189-215 (2000); Chatterjee, S. et al., “Transduction of primitive human marrow and cord blood-derived hematopoietic progenitor cells with adeno-associated virus vector,” Blood, Vol. 93, pp. 1882-1894 (1999). Transgene delivery systems have frequently included the use of the CMV immediate early promoter (Fitzsimons, H. L. et al., “Promoters and regulatory elements that improve adeno-associated virus transgene expression in the brain,” Methods, Vol. 28, pp. 227-36 (2002); Phillips, M. I., “Gene therapy for hypertension: sense and antisense strategies.” Expert Opin Biol Ther, Vol. 1, pp. 655-62 (2001); Smith, L. C. et al., “Advances in plasmid gene delivery and expression in skeletal muscle,” Curr Opin Mol Ther, Vol. 2, pp. 1504 (2000); Keating, A. et al., “Effect of different promoters on expression of genes introduced into hematopoietic and marrow stromal cells by electroporation,” Exp Hematol, Vol. 18, pp. 99-102 (1990); Muller, S. R. et al., “Efficient transfection and expression of heterologous genes in PC12 cells,” DNA Cell Biol, Vol. 9, pp. 221-9 (1990)) since it is one of the most active promoters among viral and eukaryotic species without a specific host cell type requirement. However, any number of promoters may be used in constructing the rAAV vectors of the present invention as will be recognized by one of skill in the art. For example, the rAAV-5 vector incorporates a CBA promoter.

The construction of the vectors of the present invention can be completed by widely recognized means for manufacturing AAV virions, which entails co-transfection of a host cell with two different, yet complementing plasmids. One of these contains the therapeutic or reporter transgene sandwiched between the two cis acting AAV ITRs. The AAV components that are needed for rescue and subsequent packaging of progeny recombinant genomes are provided in trans by a second plasmid encoding the viral open reading frames for rep and cap proteins. However, any number of other techniques for construction of the vectors of the present invention may be used as will be recognized by one of skill in the art. See, e.g. Gao, G. (2002) Proc Natl Acad Sci USA 99:11854-11859; Hauck, B. (2003) Journal of Virology 77(4):2768-2774; Gao, G. (2004) Journal of Virology 78(12):6381-6388. Still other methods may be used for construction of the vectors of the present invention, for example, U.S. Pat. No. 5,658,776 refers to packaging systems and processes for packaging AAV vectors that replace the AAV P5 promoter with a heterologous promoter. Alternatively. U.S. Pat. No. 5,622,856 refers to constructs and methods for AAV vector production, which provide constructs formed by moving the homologous P5 promoter to a position 3′ to the rep genes, and optionally flanking the rep-cap and repositioned P5 promoter with FRT sequences.

Furthermore, in various embodiments of the invention, the ITRs and portions of the genome of the first plasmid and the rep and cap proteins of the second plasmid can be derived from any serotype of AAV vector. In this way, the rAAV virions of the present invention can be specifically tailored to target a subject tissue with greater specificity. It is well known in the art that AAV serotype has a significant impact on tissue-specific gene expression (Hauck, B. et al., “Generation and characterization of chimeric recombinant AAV vectors,” Mol Ther, Vol. 7, pp. 419-25 (2003); Chao, H. et al., “Several log increase in therapeutic transgene delivery by distinct adeno-associated viral serotype vectors,” Mol Ther, Vol. 2, pp. 619-23 (2000); Xiao, W. et al., “Gene therapy vectors based on adeno-associated virus type 1,” J Virol, Vol. 73, pp. 3994-4003 (1999); Rabinowitz, J. E. et al., “Cross-packaging of a single adeno-associated virus (AAV) type 2 vector genome into multiple AAV serotypes enables transduction with broad specificity,” J Virol, Vol. 76, pp. 791-801 (2002): Alisky, J. M. et al., “Transduction of murine cerebellar neurons with recombinant FIV and AAV5 vectors,” Neuroreport, Vol. 11, pp. 2669-73 (2000); Chiorini, J. A. et al. “Cloning and characterization of adeno-associated virus type 5,” J Virol, Vol. 73, pp. 1309-19 (1999); Davidson, B. L. et al., “Recombinant adeno-associated virus type 2, 4, and 5 vectors: transduction of variant cell types and regions in the mammalian central nervous system,” Proc Nat Acad Sci USA, Vol. 97, pp. 3428-32 (2000); Rutledge, E. A. et al., “Infectious clones and vectors derived from adeno-associated virus (AAV) serotypes other than AAV type 2,” J Virol, Vol. 72, pp. 309-19 (1998)). For example, the DNA element of the first plasmid may be derived from one AAV serotype, the rep proteins may be derived from another AAV serotype, and the cap proteins may be derived from still another AAV serotype. In particular, the AAV vector genome can be pseudotyped by packaging with capsids from different AAV serotypes, which has been effective in directing rAAV gene therapies to specific tissues (Weitzman, M. et al., “Breaking the barriers to global gene delivery,” Nature Biotechnology, Vol. 23, Issue 3, pp. 305-306 (2005); Wang, Z. et al., “Adeno-associated virus serotype 8 efficiently delivers genes to muscle and heart,” Nature Biotechnology, Vol. 23, Issue 3, pp. 321-328 (2005); Wang, L. et al., “Sustained correction of disease in naive and AAV2-pretreated hemophilia B dogs: AAV2/8-mediated, liver-directed gene therapy,” Gene Therapy. Vol. 105, Issue 8, pp. 3079-3086 (2005)). In various embodiments of the present invention, capsids derived from AAV serotypes 1, 8, 9 and 10 may be particularly effective in intramuscular injections. Further, capsids derived from AAV serotypes 1, 7 and 8 may be particularly effective for hematopoietic stem cell transduction.

In some embodiments of the invention, rAAV virions including heterologous DNA corresponding to an ApoA-1 Milano coding sequence are generated by any conventional technique known in the art. By way of example, the recombinant AAV virions of the present invention, including the ApoA-1 Milano DNA of interest, can be produced by a standard methodology that generally involves the steps of: (1) introducing an AAV vector plasmid into a host cell; (2) introducing an AAV helper construct into the host cell, where the helper construct includes AAV coding regions capable of being expressed in the host cell to complement AAV helper functions missing from the AAV vector; (3) introducing one or more helper viruses and/or accessory function vectors into the host cell, wherein the helper virus and/or accessory function vectors provide accessory functions capable of supporting efficient rAAV virion production in the host cell; and (4) culturing the host cell to produce rAAV virions. The AAV vector, AAV helper construct and the helper virus or accessory function vector(s) can be introduced into the host cell either simultaneously or serially, using standard transfection techniques. Any number of other approaches may also be used, as will be readily recognized by one of skill in the art.

AAV vectors are constructed using known techniques to at least provide, as operatively linked components in the direction of transcription, (a) control elements including a transcriptional initiation region, (b) the ApoA-1 Milano DNA of interest and (c) a transcriptional termination region. Moreover, any coding sequence sufficiently homologous to the ApoA-1 Milano coding sequence so as to exhibit functional properties substantially similar to the ApoA-1 Milano coding sequence may be used in connection with alternate embodiments of the present invention. The control elements are selected to be functional in the targeted cell(s). The resulting construct, which contains the operatively linked components, may be bounded (5′ and 3′) with functional AAV ITR sequences. The nucleotide sequences of AAV ITR regions are known. See, e.g., Kotin, R. M. (1994) Human Gene Therapy 5:793-801; Berns, K. I., “Parvoviridae and their Replication” in Fundamental Virology, 2^{nd} Edition, (B. N. Fields and D. M. Knipe, eds.) for the AAV-2 sequence. AAV ITRs used in the vectors of the invention need not have a wild-type nucleotide sequence, and may be altered (e.g., by the insertion, deletion or substitution of nucleotides). Additionally, AAV ITRs may be derived from any of several AAV serotypes, including, without limitation, AAV-1, AAV-2, AAV-3, AAV-4. AAV-5, AAV-7, AAV-8, AAV-9, AAV-10 and the like. See, e.g. Gao et al., J. Virol. 2004 June; 78(12):6381-8; Weitzman, M. et al. (2005); Wang, Z. et al. (2005); and Wang, L. et al. (2005). Furthermore, 5′ and 3′ ITRs that flank a selected nucleotide sequence in an AAV expression vector need not necessarily be identical or derived from the same AAV serotype or isolate, so long as they function as intended (i.e., to allow for excision and replication of the bounded ApoA-1 Milano nucleotide sequence of interest).

The rAAV genome encoding the ApoA-1 Milano transgenes within AAV ITRs may be packaged in virion capsids derived from any AAV serotype including AAV-1, AAV-2, AAV-4, AAV-5, AAV-6, AAV-7, AAV-8, AAV-9, AAV-10 and the like. See, e.g. Gao et al. (2004); Weitzman, M. et al. (2005); Wang, Z. et al. (2005); and Wang, L. et al. (2005).

The virions described above are useful for treating Alzheimer's disease or to slow down the progression of Alzheimer's disease and thus are useful for the manufacture of pharmaceutical compositions which contain an effective amount of rAAV-ApoA-1 Milano vectors in admixture with inorganic or organic, solid or liquid, pharmaceutically acceptable carriers. Thus, another aspect of this invention is a composition for treating Alzheimer's disease or to slow down the progression of Alzheimer's disease described herein in combination with a pharmaceutically acceptable excipient.

The pharmaceutical compositions according to the invention are those which are suitable for oral, transdermal, topical, or parenteral, such as intramuscular or intravenous, administration to humans, and which contain the pharmacologically active rAAV transfected vectors together with a pharmaceutically acceptable carrier. The dosage depends on various factors such as the age, weight, severity of vascular condition, and other factors a doctor might identify.

In certain embodiments, the therapeutic compositions can be administered via suppository, or in tablet or capsule formulations for oral delivery. Oral formulations usually include such normally employed additives such as binders, fillers, carriers, preservatives, stabilizing agents, emulsifiers, buffers and excipients as, for example, pharmaceutical grades of mannitol, lactose, starch, magnesium stearate, sodium saccharin, cellulose, magnesium carbonate, and the like. These compositions take the form of solutions, suspensions, tablets, pills, capsules, enterics, sustained release formulations, powders, and the like. Oral formulations for gene therapy are known in the art. See, e.g. Chen, J. et al. (2004) World J. Gastroenterol 10(1):112-116. Further, other oral formulations are contemplated for use in the present invention as will be recognized by one of skill in the art.

Additional formulations which are suitable for other modes of administration, such as transdermal and topical administration, include salves, tinctures, creams, lotions, transdermal patches, transplanted skin, genetically engineered skin, stent coatings and suppositories. For salves and creams, traditional binders, carriers and excipients may include, for example, polyalkylene glycols or triglycerides. In certain embodiments, a transdermal patch may be used for delivering therapeutics. See, e.g. U.S. Pat. No. 4,638,043. Transdermal and topical formulations for gene therapy are known in the art. See, e.g. Jensen, T G (2004) Expert Opin Biol Ther. 4(5):677-82. Further, other transdermal and topical formulations are contemplated for use in the present invention as will be recognized by one of skill in the art.

Suitable dosage forms for parenteral administration can include sterile aqueous solutions of the pharmacologically active rAAV transfected vectors in water-soluble form, for example, a water-soluble salt, or sterile aqueous injection suspensions which contain substances increasing the viscosity, for example, sodium, carboxymethyl cellulose, sorbitol and/or dextran, and optionally stabilizers. In addition, the pharmacologically active rAAV transfected vectors, with or without adjuvants, can also be in lyophilized form and brought into solution prior to parenteral administration by the addition of suitable solvents.

Generally, an injectable composition of the invention may be a solution that is ready for injection, or a dry soluble composition that is ready to be combined with a solvent just prior to use, or a liquid concentrate ready for dilution prior to administration. In preparing a composition for injection strict attention must be paid to tonicity adjustment to avoid irritation.

The vehicle normally has no therapeutic activity and is nontoxic, but presents the pharmacologically active rAAV transfected vectors to the body tissues or circulation in a form appropriate for absorption. Absorption normally will occur most rapidly and completely when the pharmacologically active rAAV transfected vectors is presented as an aqueous solution. However, modification of the vehicle with water-miscible liquids or substitution with water-immiscible liquids can affect the rate of absorption. In preparing the compositions which are suitable for subcutaneous injection, one can use aqueous vehicles, water-miscible vehicles, and nonaqueous vehicles. Certain aqueous vehicles are recognized officially because of their valid use in parenterals generally.

Water-miscible vehicles are also useful in the formulation of the parenteral composition of this invention. These solvents are used primarily to affect the solubility of the pharmacologically active rAAV transfected vectors. These solvents may include, for example, ethyl alcohol, polyethylene glycol and propylene glycol.

Additional substances may be included in the injectable compositions of this invention to improve or safeguard the quality of the composition. Thus, an added substance may affect solubility, provide for patient comfort, enhance the chemical stability, or protect preparation against the growth of microorganisms. Thus, the composition may include an appropriate solubilizer, substances to make a solution isotonic, substances to act as antioxidants, and substances that act as a preservative to prevent the growth of microorganisms. These substances will be present in an amount that is appropriate for their function, but will not adversely affect the action of the composition as a treatment for disease conditions as contemplated herein.

Generally, the sterile, parenterally injectable composition of this invention and other therapeutic formulations suitable for delivery to a mammal in accordance with various embodiments of the present invention can be readily prepared by routine experimentation by the skilled artisan. Guidance as to suitable pharmaceutical formulations is provided by Remington: The Science and Practice of Pharmacy 19^{th} Ed.

In accordance with an embodiment of the invention, the rAAV virions encoding ApoA-1 Milano are delivered to a mammal in a sufficient quantity and by a sufficient delivery route so as to effect gene transfer. This provides an effective way for treating Alzheimer's disease or a symptom of Alzheimer's disease or to slow down the progression of Alzheimer's disease in mammals. In various embodiments, a sufficient and therapeutic quantity may be from about 1×10¹⁰ vector genome/kg to about 1×10¹⁴ vector genome/kg of rAAV-ApoA-1 Milano vectors in vivo. In one embodiment of the present invention, the ApoA-1 Milano vector may be delivered to a subject by first transducing multipotent stem cells (e.g. bone marrow cells, blood stem cells, stromal cells, mesenchymal stem cells etc.) with a quantity of the rAAV-ApoA-1 Milano vector, and then transplanting these cells into a mammal. In an alternate embodiment, the rAAV-ApoA-1 Milano vector may be introduced into a mammal by direct intramuscular or intravenous injection. These result in secretion of ApoA-1 Milano directly into the circulation. Further, the rAAV virions of the present invention can be delivered as a single administration or as a treatment regimen, e.g., daily, weekly, or at any other suitable time interval, as will be readily recognized by one of skill in the art. In another embodiment of the present invention, one serotype of rAAV virion can be delivered as a single administration followed by delivery of a different serotype of rAAV virion.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

### Construction of Recombinant Adeno-Associated Virus Vectors

The construction of the rAAV vectors of the present invention are completed by co-transfecting a host cell with two different plasmids. rAAV virions are prepared with the plasmids derived from various AAV serotypes. In each of the first plasmids, ApoA-1 Milano is sandwiched between the two cis acting AAV ITRs. The AAV rep and cap proteins are provided in trans by a second plasmid encoding the viral open reading frames for rep and cap proteins of AAV. In one virion, rAAV2, the first plasmid genome is derived from AAV serotype 2 and the second plasmid is derived from AAV serotype 2 (Rep2Cap2). In a second virion, rAAV5, the first plasmid genome is derived from AAV serotype 5 and the second plasmid is derived from AAV serotype 5 (Rep5Cap5). In a third virion, rAAV1, the first plasmid genome is derived from AAV serotype 2 and the second plasmid is derived from AAV serotypes 2 and 1 (Rep2Cap1). In a fourth virion, rAAV7, the first plasmid genome is derived from AAV serotype 2 and the second plasmid is derived from AAV serotypes 2 and 7 (Rep2Cap7). In a fifth virion, rAAV8, the first plasmid genome is derived from AAV serotype 2 and the second plasmid is derived from AAV serotypes 2 and 8 (Rep2Cap8). In a sixth virion, rAAV9, the first plasmid genome is derived from AAV serotype 2 and the second plasmid is derived from AAV serotypes 2 and 9 (Rep2Cap9). Other virions may be readily implemented as part of the present invention, as will be recognized by one of skill in the art.

### Example 2

### Production of Recombinant Adeno-Associated Virus (rAAV)

### Vectors:

A rAAV viral vector plasmid was constructed based on vectors previously constructed and utilized in the inventor's laboratory for the purpose of Apo A1 Milano expression. The specific rAAV vector serotypes used in this study contain each AAV serotype 2 and 8 viral capsid and a single-stranded DNA containing AAV2 inverted terminal repeat and encoding the human Apo A1 Milano gene cDNA driven by a cytomegalovirus (CMV) immediate-early promoter/enhancer. In addition, the enhanced green fluorescent protein (EGFP) marker gene was also included in the constructs to simplify the monitoring procedure for transgene detection.

### Cultured Cells:

NautCells™ (MICROBIX BIOSYSTEMS INC., Canada), a reliable and traceable 293 human embryo kidney (HEK) cell clone producing a high titre of rAAV vectors, were grown and maintained in high glucose DMEM (INVITROGEN) culture medium containing 10% fetal bovine serum. 100 units/ml-100 mg/ml penicillin-streptomycin in 5% CO₂ at 37° C.

Transfection of rAAV Using Effecten Transfection Reagent (QIAGEN):

Sub-cultured actively growing NautCells™ were placed in 15 cm culture dishes with high glucose DMEM and incubated in 5% CO₂ at 37° C. overnight. The medium was changed the next day and used for transfection 2-4 h. A plasmid mixture consisting of 4 ug of rAAV vector (individual constructs), 4 ug of AAV packaging plasmid XX2(AAV rep2 and cap2) or p5E18-VD287(AAV rep2 and cap8), and 12 mg of adenovirus helper plasmid XX6-80 were mixed with EC buffer (QIAGEN INC., Valencia. CA) to a final volume of 700 ul. Enhancer (120 ul; QIAGEN INC.) was added to each tube and vortexed immediately for 10 s. The tubes were placed at room temperature for 10 min. Fresh DMEM culture medium (4 ml) was added to each individual tube and mixed by pipetting up and down three times. The medium was then laid drop-wise onto NautCell™ while the dish was gently swirled. Transfected NautCells™ were scraped with a cell lifter at 66-72 h post-transfection in the presence of medium. The cells from five dishes were combined in a 50 ml disposable centrifuge tube, collected by spinning in SORVALL TC centrifuge at 1,000 rpm for 8 min at RT. The media are discarded, and the cell pellets were stored at −80° C. for later use.

Purification of rAAV Virus Using Discontinuous Iodixanol Density Gradients:

The cell pellets were resuspended in 1.5 ml of 150 mM NaCl, 50 mM Tris-HCl, pH 8.5. The cells subjected to five cycles of freezing (dry ice-ethanol bath) and thawing (37° C. water bath) with vortexing for 30 s after each thawing. The lysed cells were incubated with 0.5% deoxycholate (FLUKA) in the presence of 50 u/ml Benzonase (SIGMA) at 37° C. for 30 min. The lysate was clarified and recovered by centrifugation at 4500 g at 4° C. for 20 min. Purification of rAAV particles was accomplished by discontinuous iodixanol density gradient centrifugation method as previously described by Muzyczka et al. [2]. The virus was concentrated and desalted by centrifugation through the Amicon ultre-15 centrifugal filter devices (Millipore 100K NMWL device).

Dot Blot Hybridization for Determining rAAV Vector Genome (Vg) Titers and rAAV Transduction Assay for Determining Transducing Units (Tu):

rAAV vector genome titers were determined by dot-blot assay using RNA Detector Northern Blotting Kit (KPL) according to the manufacturer's instructions, and the titers were rAAV2/2 6.2×10e12 genome copies/ml, rAAV2/8 5.6×19e12 genome copies/ml. Viral transducing units (Tu) were measured by transduction of 293 cells in the presence of adenovirus helper with MOI followed by FACS.

### Example 3

### Tissue Biodistribution of Transgene Expression

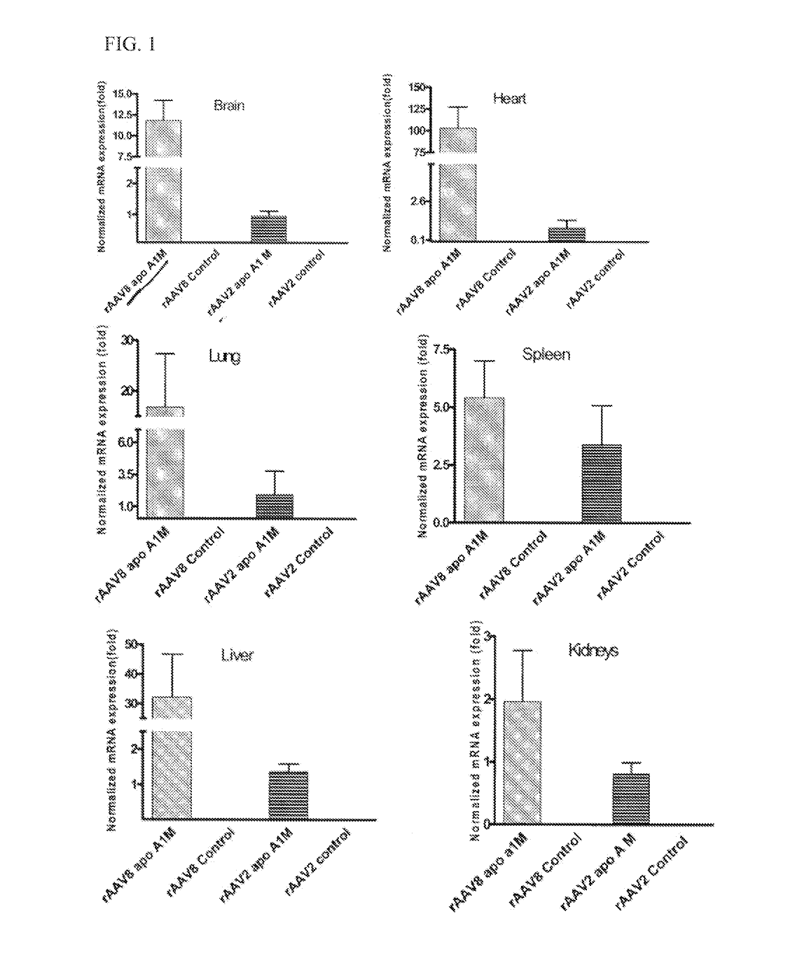

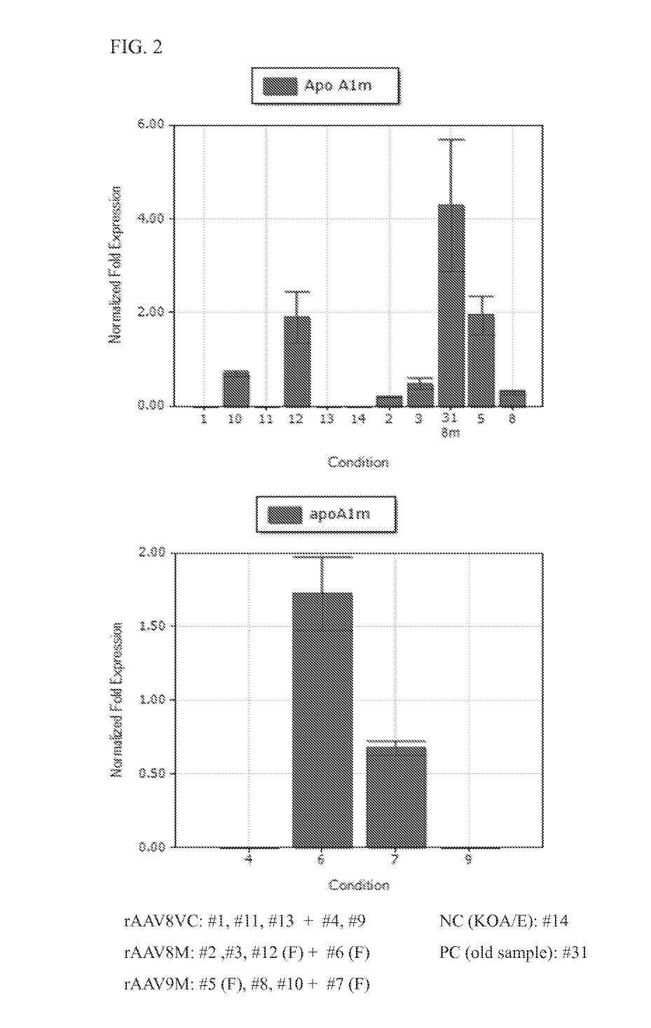

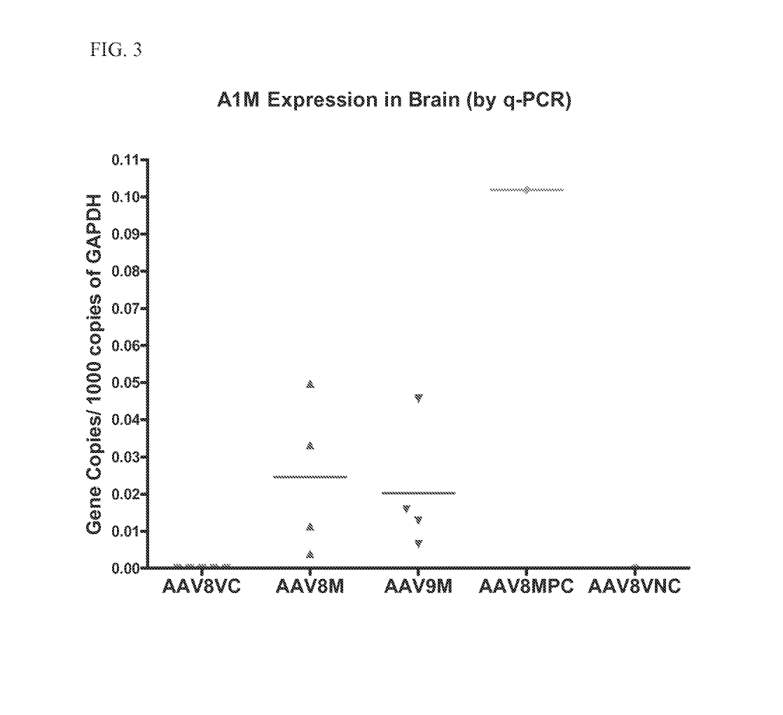

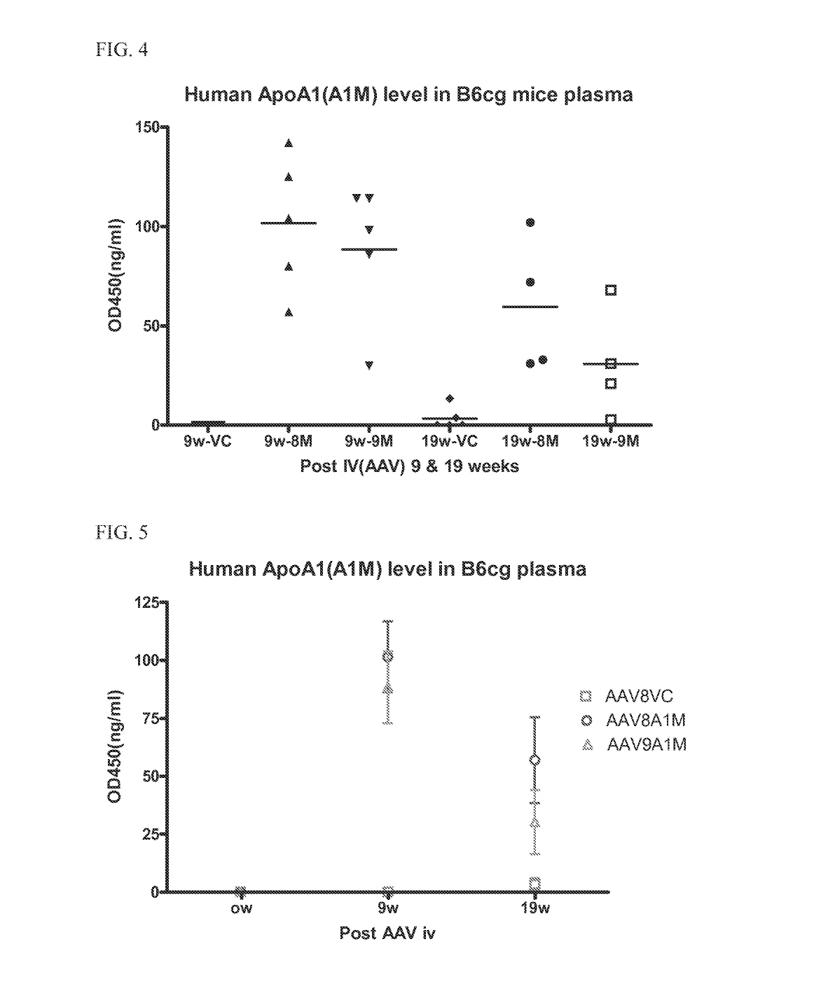

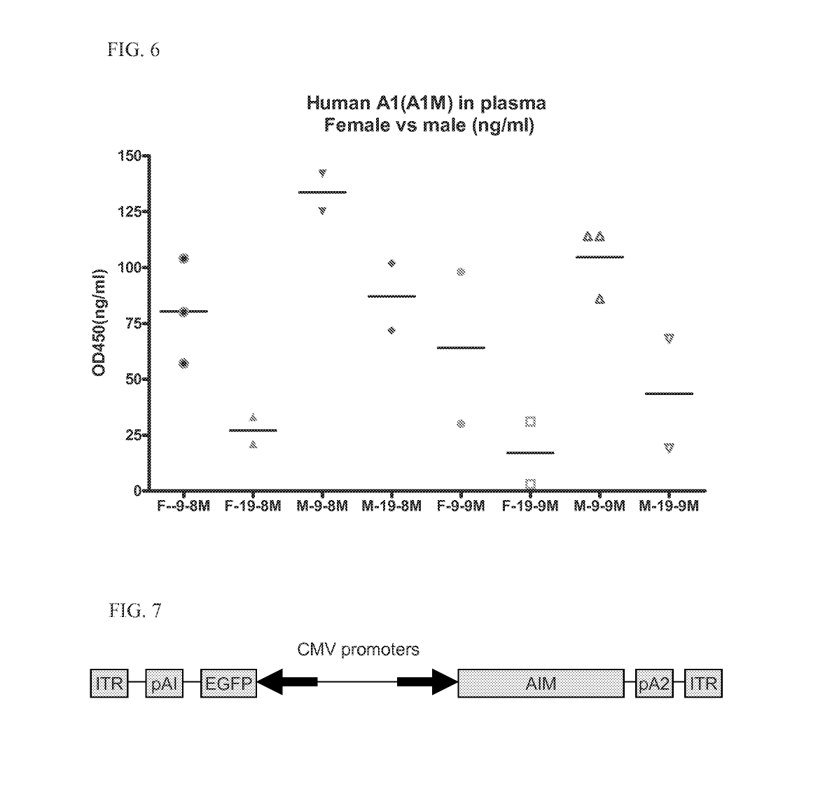

Because vector doses were identical among all the groups, a comparative analysis of rAAV transducer efficacies was possible in several organs in two serotypes. At 20 weeks after vector administration, a single mouse was killed for each rAAV vector group and total RNA was extracted from brain, lung, heart, liver, spleen, kidney and muscle. The biodistribution of transgene was performed to compare the extent of Apo A1 Milano expression in the group treated with rAAV8 (n=3) and rAAV2 (n=3) by real-time PCR. Data showed a significantly higher level of rAAV8 mediated transgene expression in the brain (11.85±2.4 vs. 0.95±0, p<0.05), heart (102.3±24.20 vs. 0.9±0.5, p<0.001), Liver (32.14±14.56 vs. 1.37±0.22, p=0.05), lung (16.49±10.75 vs. 1.86±1.8, p=0.25), spleen (5.41±1.59 vs. 3.39±1.69, p=0.22) and kidney (1.96±0.8 vs. 0.81±0.18, p=0.119) with rAAV8 Apo A1 Milano compared to rAAV2 Apo A1 Milano (FIG. 1). This indicated that rAAV8 treatment elicited higher widespread gene transfer in differential tissues than rAAV2. The biodistribution of the AAV8 serotype was interesting in that it demonstrated a wide tissue distribution. Also rAAV8 mediated more efficient Apo A1 Milano expression than rAAV2 with same titer of viral vector.

### Example 4

Viruses used in this study were (a) rAAV 8-EGFP-Vector only (3F+3M); (b). rAAV 8-EGFP-humanA1-Milano (3F+3M); and (c) rAAV9-EGFP-humanA1-Milano (2F+3M).

The amounts of rAAV for each animal were: Genome copies (DNA Particles): 1.5Ê12/per animal.

The method to deliver the rAAV was single dose intravenous (IV) injection (Tail vein injection).

The animal strain was B6Cg (Alzheimer mouse model); Male and female at about 3 months old (11-15 weeks); KO ApoE/ApoA1 mice; Female, matched age, served as negative A1 Control.

The mice (1) at the age of 11-15 weeks old had received single dose rAAV IV, (1.5E DNA particles/300 ulPBS/per mouse); (2) at the age of about 5 months old (post IV 9 weeks), mice were bled once (by retro-orbital). Plasma Apo A1-M Expression level was measured by ELISA; (3) at the end point (age of about 9 months old, post IV 19 weeks) animal were first collected blood and then following by perfusion with ice-cold 1× to collect the brain and other tissues (liver, spleen, kidney, lung, heart, thymus, blood circulated monocyte, plasma, etc.) for further study.

Plasma: ELISA was performed to detect human-A1M gene expression

Brain: RNA-RT-qPCR was performed for brain hA1M expression

### Example 5

Sequences

Human apo A1 cDNA sequences (804 bp) (SEQ ID NO: 1)
atgaaagctgcggtgctgaccttggccgtgctcttcctgacggggagccaggctcggcatttctggcagcaagatgaacccc
cccagagcccctgggatcgagtgaaggacctggccactgtgtacgtggatgtgctcaaagacagcggcagagactatgtgtc
ccagtttgaaggctccgccttgggaaaacagctaaacctaaagctccttgacaactgggacagcgtgacctccaccttcagc
aagctgcgcgaacagctcggccctgtgacccaggagttctgggataacctggaaaaggagacagagggcctgaggcaggaga
tgagcaaggatctggaggaggtgaaggccaaggtgcagccctacctggacgacttccagaagaagtggcaggaggagatgga
gctctaccgccagaaggtggagccgctgcgcgcagagctccaagagggcgcgcgccagaagctgcacgagctgcaagagaag
ctgagcccactgggcgaggagatgcgcgaccgcgcgcgcgcccatgtggacgcgctgcgcacgcatctggccccctacagcg
acgagctgcgccagcgcttggccgcgcgccttgaggctctcaaggagaacggcggcgccagactggccgagtaccacgccaa
ggccaccgagcatctgagcacgctcagcgagaaggccaagcccgcgctcgaggacctccgccaaggcctgctgcccgtgctg
gagagcttcaaggtcagcttcctgagcgctct cgaggagtacactaagaagctcaacacccagtga
Human apo A1 Protein sequence (SEQ ID NO: 2)
MKAAVLTLAVLFLTGSQARHFWQQDEPPQSPWDRVKDLATVYVDVLKDSGRDYVS
QFEGSALGKQLNLKLLDNWDSVTSTFSKLREQLGPVTQEFWDNLEKETEGLRQEMS
KDLEEVKAKVQPYLDDFQKKWQEEMELYRQKVEPLRAELQEGARQKLHELQEKLS
PLGEEMRDRARAHVDALRTHLAPYSDELRQRLAARLEALKENGGARLAEYHAKAT
EHLSTLSEKAKPALEDLRQGLLPVLESFKVSFLSALEEYTKKLNTQ

### Apo A1 Milano

The replacement of an arginine by a cysteine in codon 173 (R1173C) favors the formation of apo A1 Milano.

### PCR Primer for Human Apo A1 and Human Apo A1-M

Forward: 5′-tggatgtgctcaaagacagc-3′ (SEQ ID NO:3); Reverse: 5′-acctcctccagatccttgct-3′ (SEQ ID NO:4). In the experiments, only rAAV-hApoA1-M were injected, and thus, these primers detected hApoA1-M. These primers are specific for hApo A1 and hApo A1-M.
PCT primer for Mouse Apo A1 and Mouse ApoA1-M
Forward: 5′-tggatgcggtcaaagacagc-3′ (SEQ ID NO:5); Reverse: 5′-acttcctctaggtccttgtt-3′ (SEQ ID NO:6). These primers provide additional evidence that the primers above are specific for hApo A1 and hApo A1-M, and the primers for hApo A1 and hApo A1-M will not detect endogenous mouse Apo-A1M.

Various embodiments of the invention are described above in the Detailed Description. While these descriptions directly describe the above embodiments, it is understood that those skilled in the art may conceive modifications andior variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

What is claimed is:

1. A method, comprising:
providing a composition comprising an rAAV vector encoding ApoA-1 Milano or an active fragment thereof; and
administering the composition to a mammal in need of treatment for Alzheimer's disease, in need of slowing the progression of Alzheimer's disease, or in need of alleviating a symptom of Alzheimer's disease, to deliver the ApoA-1 Milano or an active fragment thereof to brain tissue, to treat the Alzheimer's disease, to slow the progression of Alzheimer's disease, or to alleviate the symptom of Alzheimer's disease.

2. The method of claim 1, wherein the mammal is in need of treatment for Alzheimer's disease and the method treats Alzheimer's disease.

3. The method of claim 1, wherein the mammal is in need of slowing the progression of Alzheimer's disease and the method slows the progression of Alzheimer's disease.

4. The method of claim 1, wherein the mammal is in need of alleviating the symptoms of Alzheimer's disease and the method alleviates a symptom of Alzheimer's disease.

5. The method of claim 1, wherein the rAAV vector is an rAAV8 vector encoding ApoA-1 Milano or an active fragment thereof.

6. The method of claim 1, wherein the rAAV vector is an rAAV2 vector encoding Apo A-1 Milano or an active fragment thereof.

7. The method of claim 1, wherein the rAAV vector is an rAAV9 vector encoding ApoA-1 Milano or an active fragment thereof.

8. The method of claim 1, wherein administering the composition is by intravenous injection.

9. The method of claim 1, wherein administering the composition is by intramuscular injection.

10. The method of claim 1, wherein administering the composition results in the delivery of the ApoA-1 Milano gene or a gene encoding an active fragment of ApoA-1 Milano to brain tissue.

11. The method of claim 1, wherein administering the composition results in the secretion of ApoA-1 Milano or an active fragment thereof directly into the circulation of the mammal.

12. The method of claim 1, wherein the composition comprises an rAAV8 vector encoding ApoA-1 Milano or an active fragment thereof, and administering the composition to the mammal is by intravenous injection.

13. The method of claim 1, wherein the composition comprises an rAAV9 vector encoding ApoA-1 Milano or an active fragment thereof, and administering the composition to the mammal is by intravenous injection.

14. A method of delivering ApoA1-M to brain tissue of a mammal having Alzheimer's disease or a symptom of Alzheimer's disease, comprising:
providing a composition comprising an rAAV vector encoding ApoA-1 Milano or an active fragment thereof; and
administering the composition to the mammal to deliver the ApoA-1 Milano or an active fragment thereof to the brain tissue of the mammal.

15. The method of claim 14, wherein the rAAV vector is an rAAV8 vector encoding ApoA-1 Milano or an active fragment thereof.

16. The method of claim 14, wherein the rAAV vector is an rAAV9 vector encoding ApoA-1 Milano or an active fragment thereof.

17. The method of claim 14, wherein administering the composition is by intravenous injection.

18. The method of claim 14, wherein administering the composition is by intramuscular injection.
